(19) United States
(12) Patent Application Publication
(10) Pub. No.: US 2025/0383363 A1
(43) Pub. Date: Dec. 18, 2025

(54) NEUTRALIZATION ANTIBODY ASSAY METHOD

(71) Applicant: Merck Sharp & Dohme LLC, Rahway, NJ (US)

(72) Inventors: Roy Helmy, Sayreville, NJ (US); Dilki C. Wickramarachchi, King of Prussia, PA (US); Weifeng Xu, West Windsor, NJ (US)

(73) Assignee: Merck Sharp & Dohme LLC, Rahway, NJ (US)

(21) Appl. No.: 19/114,025

(22) PCT Filed: Sep. 18, 2023
(86) PCT No.: PCT/US2023/032973

§ 371 Date: Mar. 21, 2025

Related U.S. Application Data
(60) Provisional application No. 63/376,878, filed on Sep. 23, 2022.

(51) Int. Cl. G01N 33/94; G01N 33/539; G01N 33/543; G01N 33/563; G01N 33/569; G01N 33/68

(52) U.S. Cl. CPC G01N 33/94; G01N 33/539; G01N 33/543; G01N 33/563; G01N 33/56983; G01N 33/6854; G01N 2333/165; G01N 2333/4712; G01N 2333/475; G01N 2333/525; G01N 2333/5428; G01N 2333/5443; G01N 2333/55; G01N 2333/70521; G01N 2333/70532; G01N 2333/70578; G01N 2333/7155; G01N 2333/8125

(57) ABSTRACT

This disclosure relates generally to an assay to determine the presence of neutralizing antibody (NAb) in a sample of a subject treated with a drug.

### FIELD

This disclosure relates generally to neutralizing antibody (NAb) assays, especially a method of detecting NAb in biological samples of subjects treated with a drug.

### BACKGROUND OF THE INVENTION

The approval of biological therapeutics, which are monoclonal antibody (mAb) drugs signifies the golden era of immune-regulatory cancer therapy, with approval in dozens of oncologic indications and more indications in ongoing clinical trials. However, the administration of biological therapeutics has the potential to induce undesirable immunogenicity, resulting in the development of anti-drug antibodies (ADAs), including neutralizing antibodies (NAbs). NAbs diminish therapeutic efficacy by either preventing the drug from binding to its target or inhibiting down-stream signaling upon binding due to steric hindrance. In some cases, NAbs induced by biotherapeutics cross-react and neutralize the biological activity of an endogenous counterpart, resulting in the impairment of essential normal physiological function and thus cause life-threatening adverse effects.

Due to potential broad side effects as well as treatment failure, the positivity rate and magnitude of ADA responses need to be monitored. Confirmed ADA positive samples are tested using the neutralization Ab assay to assess their neutralization ability. Due to the high dose and long half-lives of mAb biotherapeutics in oncologic indications, serum samples collected for ADA and NAb assays often contain high amounts of drug, which interferes with ADA and NAb assays.

Compared to ADA assays, drug interference is especially problematic for NAb assays. This is because labeled drug used in the ADA assay can compete with the drug present in study samples. In addition, ADA methods are usually more sensitive and additional steps such as overnight incubation or simple acid dissociation can be applied without compromising suitable assay sensitivity. In the NAb assay, however, drug from the study sample cannot be distinguished from the drug added to the NAb assay. In cases where there is a higher molar ratio of drug than NAb, the NAb will be fully complexed with the excessive amount of drug, resulting in a false negative readout. In cases where the drug is directly conjugated with horse radish peroxidase (HRP), SULFO-TAG™ or other pertinent labels, such as in indirect ligand- or cell-binding assays, the free drug from the sample will compete with the labeled drug in the assay, resulting in reduced signal thus a false positive result. In these cases, more sophisticated extraction techniques, such as Bead Extraction and Acid Dissociation (BEAD) are required to enrich NAbs and overcome drug interference. Briefly, drug/ADA complexes will be dissociated with acid then biotinylated-drugs (biotin-drug) are added to compete for NAb binding. After pulling down biotin-drug/NAb complexes with streptavidin-coated magnetic beads and washing, NAbs will be eluted with a second acid treatment, neutralized and then used in the downstream NAb assay. However, harsh acid treatment during the BEAD assay can often denature acid-sensitive Nab positive controls (PCs) and could also denature acid-labile NAbs in testing samples, leading to underestimated NAb activity [10]. In addition, the BEAD assay requires high amounts of biotin-conjugated drug in order to efficiently compete with NAbs from sample drugs, at least at 1:1 ratio of biotin-drug to sample drug, which in turn requires high amounts of streptavidin-coated magnetic beads. Both biotin-drug and Streptavidin-magnetic beads (SA-magnetic beads) are expensive reagents and manually handling magnetic beads in a 96-well format could be tedious and is one of the main concerns during assay transfer and troubleshooting.

Other cases where acid treatment failed may be due to the special format of the biotherapeutics, for example, PEGylated domain Abs, where the polyethylene glycol (PEG) portion occupies a large space surrounding the protein backbone. In this case, heating has been used to not only dissociate the ADA/drug complex but also selectively denature the domain Ab drug, due to its small molecular weight and much lower thermal stability. Denaturing the drug could facilitate the downstream biotin-drug enrichment due to reduced or eliminated competition from the denatured sample drug. However, heating to denature and dissociate has limited usage and may only apply to modalities with small molecular weights.

Therefore, there is a need for a method that overcomes the challenges mentioned above to detect neutralizing antibody in biological samples i.e., samples from patients treated with a therapeutic antibody.

### BRIEF DESCRIPTION OF THE FIGURES

FIG. 1 shows a schematic of the overview the disclosed PEG precipitation, Acid Dissociation and Biotin-drug as Assay Drug (PABAD) method. The advantages of this method over the Bead Extraction and Acid Dissociation (BEAD) method include the following: (i) only 1-step acid treatment is required. The acid treatment is milder because 1:1 ADA:drug pellet rather than the serum is acid treated (ii) biotin-labelled drug is used not just for extraction but also as the assay drug; (iii) a lower quantity of biotin-labeled drug needed compared to BEAD is required (1 mg v. 1 μg per 96 well plate); (iv) no magnetic beads are required.

FIG. 2 shows the result of screening neutralizing antibody (NAb) positive controls (PCs) for acid stability.

FIG. 3A illustrates comparison of NAb PC recovery following sample acid pretreatment for the BEAD method.

FIG. 3B illustrates comparison of PC recovery following sample acid pretreatment for the PABAD method.

FIG. 4A shows comparison of sensitivity and drug tolerance of acid sensitive NAb PC tested with BEAD in the presence of different amounts of first mAb therapeutic drug. Horizontal dashed lines indicate the cut point (the threshold value for the distinction of positive and negative results) for the BEAD method.

FIG. 4B shows comparison of sensitivity and drug tolerance of acid sensitive NAb PC tested with the PABAD method in the presence of different amounts of first mAb therapeutic drug. Horizontal dashed lines indicate the cut point for the PABAD method.

FIG. 4C shows comparison of sensitivity and drug tolerance of acid resistant NAb PC tested with the BEAD method in the presence of different amounts of first mAb therapeutic drug. Horizontal dashed lines indicate the cut point for the BEAD method.

FIG. 4D shows comparison of sensitivity and drug tolerance of acid resistant NAb PC tested with the PABAD method in the presence of different amounts of first mAb therapeutic drug. Horizontal dashed lines indicate the cut point for the PABAD method.

FIG. 5A shows comparison of sensitivity and drug tolerance of acid sensitive NAb PC tested with the BEAD method in the presence of different amounts of second mAb therapeutic drug (drug 2). Horizontal dashed lines indicate the cut point for the BEAD method.

FIG. 5B shows comparison of sensitivity and drug tolerance of acid sensitive NAb PC tested with the PABAD method in the presence of different amounts of second mAb therapeutic drug (drug 2). Horizontal dashed lines indicate the cut point for the PABAD method.

FIG. 5C shows comparison of sensitivity and drug tolerance of acid resistant NAb PC tested with the BEAD method in the presence of different amounts of second mAb therapeutic drug (drug 2). Horizontal dashed lines indicate the cut point for the BEAD method.

FIG. 5D shows comparison of sensitivity and drug tolerance of acid resistant NAb PC tested with the PABAD method in the presence of different amounts of second mAb therapeutic drug (drug 2). Horizontal dashed lines indicate the cut point for the PABAD method.

FIG. 6 illustrates the percentage occupancy by NAb on added biotin-drug. The more biotin-drug added, the lower percentage of occupancy of NAb.

FIG. 7 shows a third mAb therapeutic drug (drug 3) using the PABAD method to detect NAb in the presence of different amounts of mAb drugs. Horizontal dashed lines indicate the arbitrary cut point of 30% inhibition.

FIG. 8 shows a third mAb therapeutic drug (drug 3) using the PABAD method to detect NAb in the presence of different amounts of mAb drugs. Horizontal dashed lines indicate the arbitrary cut point of 30% inhibition.

### DETAILED DESCRIPTION OF THE DISCLOSURE

The present disclosure is directed to an assay to determine the presence of neutralizing antibody (NAb) in a sample of a subject having been treated with a drug, e.g., a therapeutic antibody.

The disclosure is directed to a method for determining the presence of absence of a neutralizing antibody (NAb) of a drug in a sample from a subject having received the drug, the method comprising the following steps: a) contacting the sample with the drug in an amount sufficient to bind any anti-drug antibody (ADA) not already bound to the drug in the sample, b) precipitating any bound ADA/drug complexes by contacting the sample in step a) with polyethylene glycol (PEG), (c) dissociating the ADA/drug complex precipitate with a mild acid solution to yield a mixture comprising free drug and free ADA, (d) contacting the mixture in step c) with the drug having a label to allow the labeled drug to bind to the free ADA to yield labeled ADA/drug complexes, (e) immobilizing the labeled ADA/drug complexes from step d) on an affinity surface, wherein the affinity surface is coated with an affinity molecule for the labeled drug, such that the affinity molecule binds to the labeled ADA/drug complex and immobilizes the ADA/drug complex to the affinity surface, (f) isolating any NAb/drug complexes from the ADA drug complexes by contacting the immobilized labeled ADA/drug complexes with the drug target having a label to allow the labeled drug target to bind to the drug in the immobilized ADA/drug complexes, and excess labeled drug target to be washed away, (g) determining the level of the labeled drug target bound to the immobilized labeled ADA/drug complexes, (h) performing steps a) to step g) of the method on a negative control (NC) that does not contain ADA and/or NAb, and (i) comparing the level of the labeled drug target bound to the immobilized labeled ADA/drug complexes in step g) performed with the sample to the level of the labeled drug target bound to the immobilized ADA/drug complexes measured in step g) performed with the NC; wherein a lower level of the labeled drug target bound to the immobilized labeled ADA/drug complexes in the sample as compared to the level of the labeled drug target bound to the immobilized labeled ADA/drug complexes in the NC indicates the presence of NAb in the sample.

In an embodiment, the method starts with contacting a sample with the drug in an amount sufficient to bind any anti-drug antibody (ADA) not already bound to the drug in the sample. An amount sufficient to bind any ADA not already bound to the drug is typically an amount of drug that is much higher than the amount of drug required to bind all the ADA molecules in the sample. In an embodiment of the method of the disclosure, the amount added range from about 10 μg/mL to about 25 μg/mL. In various embodiments of the method of the disclosure, the amount added ranges from about 5 μg/mL to about 15 μg/mL, from about 10 μg/mL to about 20 μg/mL, from about 18 μg/mL to about 25 μg/mL. In some embodiments, the amount is at least 10 μg/mL, from 10-25 μg/mL, or 25 μg/mL. The sample from a subject may comprise ADA/drug complexes and free ADA. The ADA in the sample may comprise both NAb and non-neutralizing antibody.

In one embodiment, the ADA/drug immune complexes are selectively precipitated using PEG precipitation to yield a pellet containing ADA/drug complexes where the ADA and drug are at a 1:1 ratio. Any remaining free drug is washed away with the supernatant. The pellet is then resuspended and dissociated in a mild acid for a short time at room temperature, followed by the addition of a small amount of labeled drug in base buffer, to form label drug/ADA complexes. These labeled drugs not only work as competitors for NAb binding, but they may also function as assay drugs once they are transferred and bound to the affinity plate. Labeled drug targets, are added to bind to the labeled drug (labeled drug/ADA complex) on the affinity surface. Increased amounts of NAb in the sample indicate that higher percentages of biotin-drug are bound and blocked by NAbs, hence there is lower drug-target signal. Thus, PEG precipitation not only eliminates free drug, but also the harsh first acid treatment needed for BEAD, as well as the need for high amounts of drug label.

In one embodiment of the selective precipitation of the ADA/drug complexes is performed by polyethylene glycol (PEG) precipitation, wherein the PEG used is PEG or PEG-Sodium chloride (PEG-NaCl). In various embodiments of the method, the PEG used in the selective precipitation of ADA/drug immune complexes is at a concentration of from about 1% to about 10.0%, from about 2% to about 7.0%, from about 5% to about 8%, or from about 6% to about 10% PEG. In various embodiments of the method, the PEG used in the selective precipitation of ADA/drug immune complexes is at a concentration of from about 1% to about 10.0%, about 1%, about 2%, about 3.5%, about 4%, about 4.5%, about 5%, about 5.5%, or about 6% PEG. In some embodiments of the invention, the PEG used in the selective precipitation of ADA/drug immune complexes is from about 3% to about 6% PEG. In some embodiments of the invention, the PEG used in the selective precipitation of ADA/drug immune complexes is from about 3% to about 5% PEG. In some embodiments of the invention, the PEG used in the selective precipitation of ADA/drug immune complexes is from about 4% to about 6% PEG. In some embodiments of the invention, the PEG used in the selective precipitation of ADA/drug immune complexes is from about 4% to about 5% PEG.

In various embodiments of the method, the PEG used in the selective precipitation of ADA/drug immune complexes is PEG-NaCl at the concentration of from about 1% to about 10.0%, from about 2% to about 7.0%, from about 5% to about 8%, or from about 6% to about 10% PEG-NaCl. In various embodiment s of the method the PEG-NaCl used in the selective precipitation of ADA/drug immune complexes is at the concentration of from about 1% to about 10.0%, about 1%, about 2%, about 3.5%, about 4%, about 4.5%, about 5%, about 5.5%, or about 6% PEG. In some embodiments of the invention, the PEG-NaCl used in the selective precipitation of ADA/drug immune complexes is from about 3.5% to about 4.5% PEG-NaCl. In some embodiments of the method, the PEG-NaCl used in the selective precipitation of ADA/drug immune complexes is 4% PEG-NaCl. In some embodiments of the invention, the PEG-NaCl used in the selective precipitation of ADA/drug immune complexes is from about 4% to about 5% PEG-NaCl.

In an aspect of the method of the disclosure, the PEG in the PEG or PEG-NaCl used in the selective precipitation of ADA/drug immune complexes is a PEG having a molecular weight from 1,000 to 40,000 daltons. In various embodiments of the method of the disclosure, the PEG is PEG1000, PEG1450, PEG3350, PEG 3000, PEG6000, PEG8000, PEG10000, PEG14000, PEG15000, PEG20000, or PEG25000. In some embodiments, PEG8000 is used.

In an embodiment of the method, the pellet precipitate of step b) containing a 1:1 ratio of ADA to the drug immune complex is dissociated after treatment with mild acid selected from nonlimiting examples of acids including lactic acid, citric acid, oxalic acid, trifluoroacetic acid, succinic acid, malic acid, aspartic acid, nitric acid, hydrochloric acid, sulfuric acid, boric acid, and combinations thereof. In some embodiments of the method, the acid is lactic acid. Lactic acid at the concentration of from about 20 mM to 250 mM may be used in the dissociation of the ADA/drug immune complex. In some embodiments, about 20 mM, about 50 mM, about 100 mM, about 150 mM, or about 200 mM of lactic acid may be used in the dissociation of the ADA/drug immune complex. In some embodiments of the invention, lactic acid at the concentration of from about 50 mM to 100 mM may be used in the dissociation of the ADA/drug immune complex.

In an alternate embodiment of the method the pellet precipitate from step b) containing a 1:1 ratio of ADA to the drug immune complex is dissociated after treatment with a mild base.

In an aspect, the pellet precipitate from step b) containing a 1:1 ratio of ADA to the drug immune complex is dissociated after treatment with mild acid for a time from about 10 minutes to about 60 minutes. In some embodiments, the pellet is dissociated with mild acid for about 15 minutes.

The drug, e.g., therapeutic antibodies and antigen-binding fragments disclosed herein, is conjugated to a label that binds to an affinity molecule. The labeled drug binds to the affinity surface that is coated with an affinity molecule that binds to the labeled ADA/drug complex to immobilize the ADA/drug complex to the affinity surface. Non-limiting examples of the drug label and its affinity molecule (label/affinity molecule pairs) are biotin/streptavidin, biotin/avidin, biotin/captavidin, protein A/immunoglobulin, protein G/immunoglobulin, and GST/gluthione.

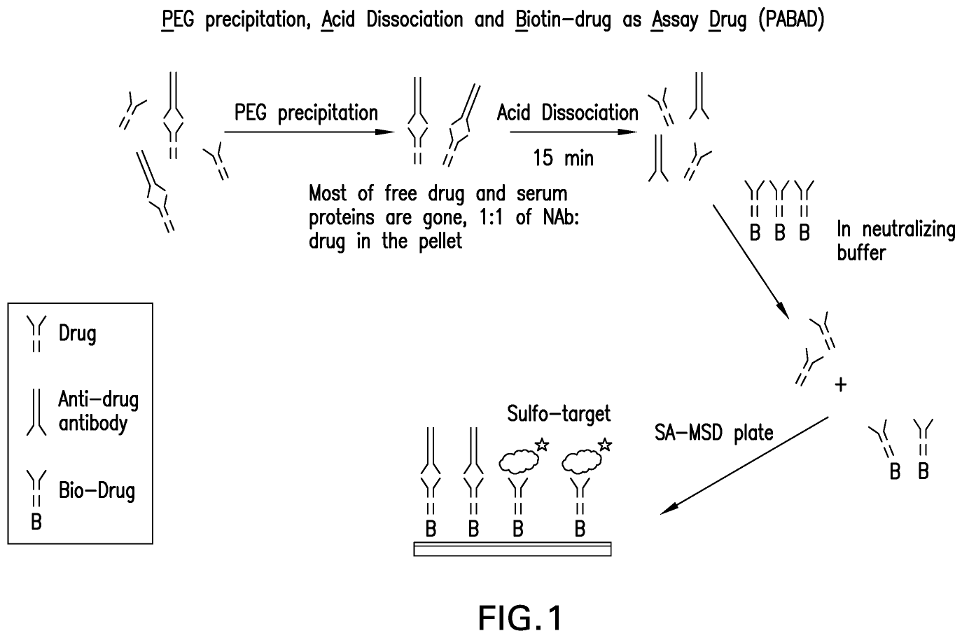

In some embodiments, the drug is labeled with biotin, and the affinity molecule is streptavidin. The method further comprises adding a small amount of labelled drug, e.g., biotin-labelled drug, between about 1 ng to about 10 ng per 96 well plate in a base buffer, which terminates the acid dissociation and allows the labelled drug to bind with the dissociated ADA/drug immune complex. Further the labelled sample is transferred to an affinity surface coated with an affinity molecule specific for the labeled drug (i.e., a molecule binds the label), for example a streptavidin (SA)-MSD plate to capture both a biotin-labelled drug bound to ADA (and free biotin-labelled drug). The captured labelled drug bound to ADA is treated with a labelled drug target and analyzed for the signal from the labelled drug target for the presence of NAb. The label-tagged drug target only binds to the antibody that is not blocked by bound NAb, i.e., a fraction of the labelled drug bound to ADA (FIG. 1).

In an embodiment of the method, the drug target is labelled by a detectable label. In some embodiments, the detectable label of the labelled drug target is selected from the group consisting of: (i) a sulfo-tag label, (ii) a chemiluminescent label, (iiii) an electrochemiluminescent label, (iv) a radioactive isotope, (v) a fluorescent label, and (vi) an enzyme label (detected by an enzymatic reaction). In some embodiments of the method, the detectable label of the labelled drug target is an electrochemiluminescent label such as a sulfo-tagged label (SULFO-TAG™).

The NAb in the sample is detected by comparing signal from the labeled drug target against negative controls (NCs) (samples with no NAb), and optionally positive controls (PCs) (samples having NAb, e.g., negative samples spiked with NAb). In an embodiment, NAb NCs are obtained by pooling commercially available serum samples from patients that have not been exposed to the drug (naïve human pooled serum or normal serum samples). The absence of the drug may in a commercially available serum be confirmed by screening the naïve serum sample for the presence of drug. PCs may be obtained by spiking the naïve serum sample with control NAbs that are specific to the therapeutic drugs that are tested.

In some embodiments, the naive pooled serum with no added control NAb added are used as NAb NCs. In some embodiments, NAb positive controls are obtained by spiking the naïve serum sample with control NAbs. Control NAbs that are specific to different therapeutic drugs may be created by methods known in the art for producing monoclonal antibodies and recombinant proteins.

NAb PCs may be obtained by spiking the naïve serum sample with 0.1-2 μg/mL of NAbs. Naïve serum may be spiked with NAb at the concentration of between about 0.2-0.5 μg/mL to obtain Low Positive Control (LPC). In some embodiments, naïve serum may be spiked with NAb at the concentration from about 0.1 to about 0.25 μg/mL, from about 0.2 to about 0.45 μg/mL, or from about 0.25 to about 0.5 μg/mL of naïve pooled serum to obtain LPC. In some embodiments, naïve serum may be spiked with 0.2 μg/mL to obtain LPC. Positive controls may be obtained by spiking the naïve serum sample with 0-2 μg/mL of control NAbs.

Naïve serum may be spiked with NAb at the concentration of from about 0.8-2 μg/mL to obtain a High Positive Control (HPC). In various embodiments, naïve serum may be spiked with NAb at the concentration of from about 0.8 to about 0.9 μg/mL, from about 0.9 to about 1 μg/mL, from about 1 to about 1.15 μg/mL, from about 1.1 to about 1.25, or from about 1.2 to about 2 μg/mL of naïve pooled serum to obtain LPC. In some embodiments, naïve serum may be spiked with 1.25 μg/mL of naïve pooled serum to obtain HPC.

A lower level of the labeled drug target bound to the immobilized labeled ADA/drug complexes in the sample as compared to the level of the labeled drug target bound to the immobilized labeled ADA/drug complexes in the NC indicates the presence of NAb in the sample. Increased amounts of NAb in the sample indicate that higher percentage of labeled-drug is bound and blocked by NAbs, and hence there is less label drug target binding and thus lower signal from the label, i.e., lower signal from the label tag indicates presence of NAb.

A signal detected from a sample with no NAb will be comparable to that signal obtained of the NAb NC. The signal detected from a sample positive for NAb would be lower than that obtained from the negative control and may be compared to the signal obtained from LPC and HPC.

In an embodiment, a cut point for percent change in the level of the labeled drug target bound to the immobilized labeled ADA/drug complexes is established by determining the level of labeled drug target in multiple samples from patients treated with the therapeutic drug. In an embodiment, multiple cut points ranging from about 5% to about 100% inhibition may be established. In various embodiments cut points of about 5%, of about 8%, of about 10%, of about 14%, of about 15%, of about 20%, of about 25%, of about 30%, of about 32%, of about 35%, of about 40%, of about 45%, of about 50%, of about 55%, of about 60%, of about 65%, of about 70%, of about 75%, of about 80%, of about 90%, of about 95%, and about 100%, may be established. A cut point may be established by testing multiple patient samples treated with therapeutic drug. In various embodiments of the method, a cut point may be established by testing about 50 to about 200 patient samples.

In some embodiments of the method, a portion of the dissociated ADA and free drug resulting from step c) is utilized for a total ADA detection assay.

In some embodiments of the method, a portion of the dissociated ADA and free drug resulting from step c) or the NAb/drug complexes from step f) are processed to detect the presence of total ADA, NAb, and/or for evaluating the activity of any NAb in a cell-based binding or functional assay. In some embodiments, to be used in a cell-based binding or functional assay, the NAb/drug complexes from step f), e.g., where the ADA is immobilized on a Streptavidin-bead (SA-BEAD) bound to a biotinylated drug, is further processed by a 2nd acid dissociation to yield free NAb to be used in cell-based binding or functional assays. Some non-limiting examples of cell-binding or functional assays are, Enzyme-linked immunosorbent assay (ELISA), Bridging ELISA, homogenous mobility shift assay (HMSA) and Cell-based reporter gene assay (RGA).

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values and ranges).

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients or steps and permit the presence of other ingredients or steps. However, the term “comprising” should be construed to include support for the more narrower embodiments of: (i) “consisting of”, which allows the presence of only the named ingredients or steps, and (ii) “consisting essentially of”, which allows the presence of only the named ingredients or steps, along with immaterial additions, e.g., any acceptable carriers or fluids.

The term “antibody,” is used in the broadest sense and specifically encompasses, for example, individual monoclonal antibodies (including neutralizing antibodies, full length or intact monoclonal antibodies), antibody compositions with polyepitopic or monoepitopic specificity, polyclonal or monovalent regions. Examples of antigen-binding fragments include, but are not limited to, Fab, Fab′, F(ab′) 2, and Fv fragments; diabodies; linear antibodies; single-chain antibody molecules, e.g., sc-Fv; multispecific antibodies formed from antibody fragments.

The drug referenced herein is an antibody and, in specific embodiments, a therapeutic antibody. The term “therapeutic antibody” refers to an antibody that is administered as a therapeutic or a drug, including a therapeutic antibody that is in development or is undergoing pre-clinical or clinical testing. In specific embodiments, the therapeutic antibody is an antibody of use in the treatment of inflammatory disease, cancer, or autoimmunity. In specific embodiments the therapeutic antibody is an anti-CD27 antibody or an antigen binding fragment thereof, an anti-LAG3 antibody or an antigen binding fragment thereof, an anti-TIGIT antibody or an antigen binding fragment thereof, an anti-VISTA antibody or an antigen binding fragment thereof, an anti-BTLA antibody or an antigen binding fragment thereof, an anti-TIM3 antibody or an antigen binding fragment thereof, an anti-CTLA4 antibody or an antigen binding fragment thereof, an anti-HVEM antibody or an antigen binding fragment thereof, an anti-CD70 antibody or an antigen binding fragment thereof, an anti-OX40 antibody or an antigen binding fragment thereof, an anti-CD28 antibody or an antigen binding fragment thereof, an anti-PD1 antibody or an antigen binding fragment thereof, an anti-PDL1 antibody or an antigen binding fragment thereof, an anti-PDL2 antibody or an antigen binding fragment thereof, an anti-GITR antibody or an antigen binding fragment thereof, an anti-ICOS antibody or an antigen binding fragment thereof, an anti-SIRPα antibody or an antigen binding fragment thereof, an anti-ILT2 antibody or an antigen binding fragment thereof, an anti-ILT3 antibody or an antigen binding fragment thereof, an anti-ILT4 antibody or an antigen binding fragment thereof, an anti-ILT5 antibody or an antigen binding fragment thereof, an anti-4-1BB antibody or an antigen binding fragment thereof, an anti-NK2GA antibody or an antigen binding fragment thereof, an anti-NK2GC antibody or an antigen binding fragment thereof, an anti-NK2GE antibody or an antigen binding fragment thereof, an anti-TSLP antibody or an antigen binding fragment thereof, an anti-IL10 antibody or an antigen binding fragment thereof. Non-limiting examples of therapeutic antibodies are avelumab, basiliximab, bevacizumab, bezlotoxumab, brodalumab, canakinumab, catumaxomab, dupilumab, durvalumab, daratumumab, elotuzumab, fanolesomab, ocrelizumab, reslizumab, olaratumab, necitumumab, infliximab, obiltoxaximab, atezolizumab, mepolizumab, nivolumab, alirocumab, idarucizumab, evolocumab, dinutuximab, pembrolizumab, ramucirumab, vedolizumab, alemtuzumab, pertuzumab, infliximab, obinutuzumab, brentuximab, belimumab, ipilimumab, denosumab, ofatumumab, besilesomab, tocilizumab, golimumab, ustekinumab, certolizumab pegol, catumaxomab, eculizumab, ranibizumab, panitumumab, natalizumab, bevacizumab, omalizumab, cetuximab, efalizumab, ibritumomab tiuxetan, adalimumab, tositumomab, alemtuzumab, trastuzumab, gemtuzumab ozogamicin, infliximab, palivizumab, necitumumab, raxibacumab, rituximab, secukinumab, siltuximab.

The term “antigen” as used herein is meant any substance that causes the immune system to produce antibodies or specific cell-mediated immune responses against it. A disease-associated antigen is any substance that is associated with any disease that causes the immune system to produce antibodies or a specific-cell mediated response against it.

The terms “label” and “detectable label” interchangeably refer to a composition detectable by spectroscopic, photochemical, biochemical, immunochemical, chemical, or other physical means. For example, useful labels include fluorescent dyes, luminescent agents, radioisotopes (e.g., ³²P, ³H), electron-dense reagents, enzymes, biotin, digoxigenin, or haptens and proteins, nucleic acids, or other entities which may be made detectable, e.g., by incorporating a radiolabel into an oligonucleotide, peptide, or antibody specifically reactive with a target molecule. Any method known in the art for conjugating an antibody to the label may be employed, e.g., using methods described in Hermanson, Bioconjugate Techniques 1996, Academic Press, Inc., San Diego.

As used herein, “the drug target,” “therapeutic antibody target,” “antibody target,” or “protein target” means the antigen that is targeted by the therapeutic antibody. Examples of a drug target that is targeted by an antibody drug include but are not limited to proteins such as TNFα, CD27, PD1, PDL1, PDL2, TIGIT, LAG-3, VISTA, BTLA, TIM3, CTLA4, HVEM, CD70, OX40, CD28, GITR, ICOS, SIRPα, IL10, IL-4R, IL-6R, IL-33, CD20, CD3, IL-33, IL-2, IL-15, IL-18 Feld 1, C5, ANGPTL-3, ACTIVIN A, GDF8, PCSK9, VEGF, Tie-2, NGF, or a viral antigens, such as SARS-cov, ebola or mers-cov.

The drug target disclosed herein may be conjugated with a detectable label. Examples of detectable labels include but are not limited to radioisotopes, chemiluminescent labels, electrochemiluminescent labels, fluorophores, chromophores, and enzyme labels. In some embodiments of the method, the label-tag is an electrochemiluminescent label. In a specific embodiment of the method is an electrochemiluminescent label-tag such as the SULFO-TAG™ (Meso Scale Discovery), that measures the signal from Ruthenium bound to a sulphocomplex.

As used herein, term “immunogenicity” or “immunoreactivity” refers to the development of an adaptive immune response to a drug or in specific embodiments the therapeutic antibody administered to a subject i.e., the antibody is recognized as foreign and generating a humoral or cellular immune response.

“Anti-drug antibodies” or “ADA” are antibodies, which may be directed against any region of the drug, like e.g., the variable domain, the constant domains, or the glycostructure of the drug. Such anti-drug antibodies may occur during antibody therapy as an immunogenic reaction of a patient (see Pan, Y., et al., FASEB J. 9 (1995) 43-49). Most of the “anti-drug antibodies” bind to one or more of the complementary determining regions of the drug. The affinity of anti-drug antibodies to their drug's antigen is in general lower compared to the affinity of the drug for its target antigen. The antidrug antibody may be neutralizing or non-neutralizing antibody.

“Neutralizing antibody” or “NAb” as used herein are a subset of ADA. Neutralizing antibodies may be the most clinically relevant portion of ADA as they bind to the active site of the therapeutic antibody, i.e., binds the antigen binding site of the therapeutic antibody preventing the therapeutic antibody from binding the therapeutic antibody target, and inhibits its pharmacological function, while non-NAb bind to a site that is not involved in target binding and which renders the drug pharmacologically active, though its clearance from circulation can still be affected (Amrani et al., Journal of Translational Autoimmunity 1 (2019) Article 100004).

The term “PEG-precipitation” or “precipitation using PEG” refers to precipitation of heavy molecular weight protein complexes, e.g., drug-anti-drug antibody complex, in a PEG solution, wherein the concentration of PEG is increased until effective protein concentration is increased until critical concentration is exceeded, and precipitation occurs.

The term “sample” includes, but is not limited to, any quantity of a substance from a living thing or formerly living thing. Such living things include, but are not limited to, humans, mice, monkeys, rats, rabbits, and other animals and such samples may include, but are not limited to, whole blood, serum or plasma from a subject. The sample may be for example a serum sample, obtained from a subject during clinical or preclinical testing of a therapeutic antibody.

The term “subject” as used herein refers to a human or non-human organism. The methods described herein refer to both human and animal subjects. Subjects can be “patients,” receiving a therapeutic antibody as treatment, and may also include participants in clinical trials for a drug, wherein the subject has been administered the drug for trial purposes.

### Abbreviations for Examples

Ab Antibody
ADA Anti-drug antibodies
ADA/drug Antidrug antibody drug complex
BEAD Bead Extraction and Acid Dissociation
BSA Bovine Serum Albumin
CLB Competitive Ligand Binding
FBS Fetal Bovine Serum
Fc Fragment Crystallizable
HCl Hydrochloric Acid
hFc Human Fc
HPC High Positive Control
LA Lactic Acid
LBD ligand Binding Assay
LPC Low Positive Control
M Molar
Mab Monoclonal Antibody
mFc Mouse Fc
MRD Minimum Required Dilution
N Normal
NAb Neutralizing Antibody
NaCl Sodium Chloride
NC Negative Control
NHS N-Hydroxysuccinimide
PandA Precipitation and Acid Dissociation
PABAD PEG precipitation, Acid Dissociation and Biotin-drug as Assay Drug
PBS Phosphate Buffered Saline
PBST Phosphate Buffered Saline with Tween
PC Positive Control
PEG Polyethylene Glycol
RPM Revolutions Per Minute
RT Room temperature
SA Streptavidin
S/N Signal/Noise

### EXAMPLES

The following examples are meant to be illustrative and should not be construed as further limiting. The contents of the figures and all references, patents, and published patent applications cited throughout this application are expressly incorporated herein by reference.

### Materials and Methods

### Reagents

Monoclonal antibody therapeutics and anti-drug antibody clones that were used as positive controls were all developed by Merck & Co., Inc. (San Francisco, CA, USA). Naïve human pooled serum and serum from individual donors were purchased from BioIVT (Hicksville, NY). Different clones of anti-His antibodies were either developed by Merck & Co., Inc. (San Francisco, CA, USA) or purchased from R&D systems Inc., (Minneapolis, MN). Anti-mouse Fc antibody and anti-human Fc antibody was purchased from Invitrogen, Waltham, MA. Target protein containing His-tag, human Fc or mouse Fc were all purchased from Acrobiosystems, Newark, DE. Small scale in-house sulfo-tagged protein labeling was carried out using MSD Gold Sulfo Tag NHS ester (2 μmol) (Meso Scale Diagnostic, Rockville, MD) and Zeba spin desalting columns 40K from Thermo Scientific, Waltham, MA. PEG8000 and 20% PEG-NaCl were purchased from TekNova (Hollister, CA). V bottom plates used for precipitation were from Nunc, Rochester, NY. The acids tested were prepared using lactic acid (Spectrum Chemical MFG Corp, New Brunswick, NJ), Glycine (GE Healthcare Life Sciences, Piscataway, NJ), Glacial Acetic acid (Fisher chemicals, Pittsburgh, PA) and 1 Normal (N) Hydrochloride acid (used for pH adjustments) from Fisher chemicals (Pittsburgh, PA). Neutralizing buffers, 1 molar (M) Tris-HCl pH 8.8 and 1.5 M Tris-HCl pH 8.8 were purchased from TekNova (Hollister, CA). All buffers used in these experiments were prepared using FBS (Sigma-Aldrich Inc, St. Louis, MO), BSA (bioWORLD, Dublin, OH), PBS, PBST and distilled water are all from Merck & Co., Inc. (West Point, PA, USA). Biotin-drug conjugation and sulfo-tagged drug preparations were completed at Radix (Georgetown, TX), while large-scale batches of sulfo-tagged target protein was prepared at Syneos Health (Princeton, NJ). Streptavidin plates (SA-MSD plates), High Bind with hydrophilic surface (HB-MSD plate) and Read bufferT (4×) were all from Meso Scale Diagnostic (Rockville, MD).

### Bead Extraction and Acid Dissociation (BEAD) Procedure

Briefly, 50 μL of human serum samples and controls were first mixed with an equal volume of 400 mM glycine-HCl, at pH 2.0, and incubated at RT for 60 min on a shaker (Labnet Orbit P4, Woodbridge, NJ) at 900 revolutions per minute (rpm). To each sample, 11.2 μL 1.5 M Trizma Base (pH 8.8) containing 89 μg/mL biotin-drug was added and incubated overnight while shaking at 900 rpm. NAbs, dissociated from drug product, would bind to biotin-drug and were then immobilized on 20 μL streptavidin-coated magnetic beads added at 6 mg/mL. Bead-complexes were then captured using a microplate magnet (96 F Magnet LifeSep Biomagnetic Separators, BioTek, Winooski, VT) and washed three times with PBST. Bound NAbs were released from the bead complex by a second acid treatment with 80 μL of 200 mM glycine at pH 2.0, for 10 min shaking at 900 rpm at RT. Eighty microliters of this NAbs-containing acid solution was then transferred to a new plate and neutralized with 15 μL of 10% FBS in IM Tris Hydrochloric acid (HCl) pH 8.8.

### PABAD Procedure

Excess free drug (60 μg/mL) in assay buffer was added to samples (50 μL serum+10 μL drug/buffer) and incubated for 1 h at 37° C. with shaking at 450 rpm in a V bottom polypropylene plate. This step allowed NAb-drug complex formation for any remaining free NAbs in the samples [12]. Following complex formation, 40 μL of 10% PEG-NaCl solution was added to each sample, (the final concentration of PEG-NaCl in the samples was 4%), incubated for 5 min at RT while shaking at 450 rpm, then incubated overnight at 4° C. without shaking. The NAb-drug precipitation conditions using different PEG or PEG-NaCls were optimized to achieve the preferred specificity and sensitivity of the assay and to minimize the impact of other serum components, particularly non-specific serum proteins.

Following overnight incubation, the plates were centrifuged at 3700 g for 30 mins at 4° C. The supernatant was removed carefully without disturbing the pellet in the center of V bottom well while tilting the plate. The samples were washed twice by adding 200 μL of 4% PEG-NaCl solution, followed by centrifugation at 3700 g at 4° C. for 20 mins. After the last wash, the supernatant was carefully removed. Then the samples were treated with 150 μL of 50 mM LA for 15 mins at RT while shaking at 600 rpm. Afterwards, biotin-drug (the final concentration in samples was 20 ng/mL) was added to samples in neutralizing buffer and incubated for 90 mins at RT while shaking at 600 rpm. Once the incubation with the biotin-drug was completed, 50 μL of the samples were added in duplicate to a SA-MSD plate that was previously blocked with 10% FBS in PBS and incubated for an hour at RT while shaking at 450 rpm. SA-MSD plates were then washed 3 times with PBS, tap dried and incubated with 50 μL of sulfo-tagged target (0.25 μg/mL) for an hour at RT while shaking at 450 rpm. Upon completion, the plates were washed 3 times with PBS and tap-dried to remove any remaining buffer. Read buffer T (Tris-based buffer containing tripropylamine) (2×) (150 μL) was added to the samples and plates were read using an MSD plate reader. Samples in the absence of any Positive Control (PC) were considered full assay responses without any inhibition. Percent inhibition was calculated by subtracting the ratio of samples response in the presence of PC by full assay response from 1 and multiplying by 100 ((1−S(sample reading)/Control (reading)×100).

### Example 1

### Overview of the PABAD Assay Format

An overview of the PABAD method is shown in FIG. 1 with black arrows indicating the steps. First, additional drug material is added to the samples to make sure all ADA (ADA comprises NAbs and non-neutralizing ADA) in the samples will be bound with drug to form ADA/Drug immune complexes. PEG-NaCl is then added to the sample at an optimal concentration to selectively precipitate immune complexes, but not free drugs. After overnight incubation at 4° C. without agitation, immune complexes will be precipitated by centrifugation, washed and then reconstituted with an acid solution. Biotin-drugs are then added in the neutralization buffer so that ADA/NAb can now bind to the biotin-drug. After transferring the mixture to a streptavidin-MSD plate and washing, all free drugs will be gone and only biotin-drug will be bound to the plate and free biotin-drug can be detected with sulfo-tagged drug targets.

### Example 2

### Feasibility of the Competitive Ligand Binding (CLB) NAb Assay Format

The most common format for a CLB NAb assay is to coat recombinant drug targets on the plate and then add HRP- or ruthenium-labeled drug preincubated with serum samples. If samples contain NAb, then the binding of the drug to the coated drug target is reduced to give a lower signal compared to control samples which lack NAb. Our proposed method, however, adds biotin-drug to the acid dissociated drug/NAb pellet to compete for NAb binding. It would be optimal for the mixture of biotin-drug/drug/NAb to be added to the SA-coated MSD, so that only biotin-drug will bind to SA-MSD while free drug will be washed away.

To test the best condition for this assay format, a fixed amount of Biotin-drug was coated to a SA-MSD plate, followed by either direct detection with various serially diluted sulfo-tagged target proteins (recombinant, mouser Fc (mFc)-fused, human Fc (hFc)-fused, His-tagged), or indirectly with His-tagged target protein or mFc-fused target protein. Then, these were detected using different sulfo-tagged anti-His clones or anti-mFc antibodies. As shown in Table 1, different formats yielded drastically different signal-to-noise (S/N) ratios. Sulfo-tag labeled His-target protein was selected as the detection reagent in the CLB NAb assay due to the ease of the assay workflow and high enough S/N (Table 1).

TABLE 1
S/N ratio of sulfo-tagged proteins in different assay formats

| | | | | | Assay format 2 | | | |
|---|---|---|---|---|---|---|---|---|
| | Assay format 1 | | | | Anti- | Anti- | Anti- | Anti- |
| Concentration | | mFc- | hFc - | His- | his | his | his | mFc |
| (ng/mL) | target | target | target | target | clone 1 | clone 2 | clone 3 | Ab |
| 6000 | 9616 | 28311 | 23886 | 32218 | 12364 | 2393 | 2287 | 218 |
| 2400 | 3302 | 5091 | 1693 | 22410 | 12010 | 2293 | 2039 | 219 |
| 960 | 263 | 2108 | 701 | 9136 | 11755 | 1838 | 1405 | 217 |
| 384 | 41 | 642 | 183 | 3484 | 10449 | 865 | 607 | 211 |
| 153 | 17 | 293 | 72 | 1299 | 4420 | 345 | 237 | 128 |
| 61 | 8 | 125 | 33 | 487 | 1560 | 127 | 85 | 58 |
| 24 | 6 | 67 | 17 | 198 | 618 | 48 | 33 | 24 |
| 0 | 1 | 1 | 1 | 1 | 1 | 1 | 1 | 1 |

Ab: Antibody;
S/N: Signal/Noise;
mFc: mouse Fc;
hFc: human Fc
S/N ratio was calculated for each protein.

### Example 3

### Feasibility of the Competitive Ligand Binding (CLB) NAb Assay Format

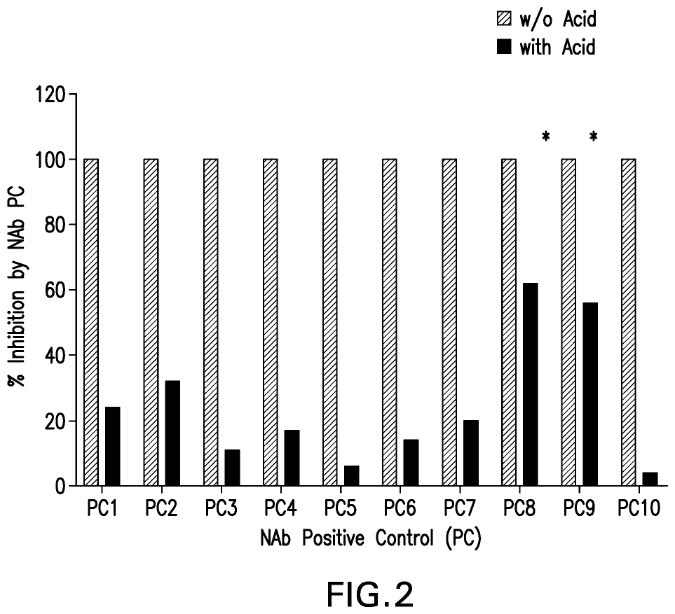

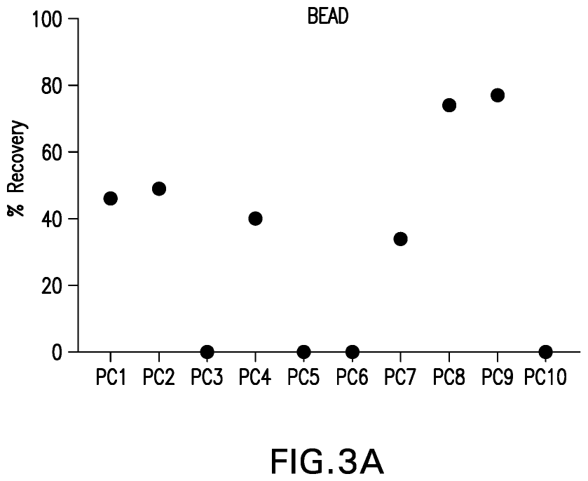

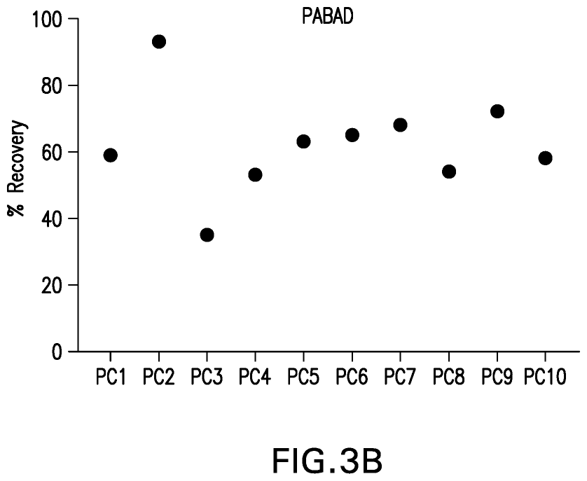

One of the potential advantages of PABAD over BEAD is reduced acid treatment, hence the possibility of better compatibility with acid-sensitive NAb PCs. To test this hypothesis, we first tested acid stability of the 10 NAb PCs available for this project. NAb PCs were first incubated with 600 mM acetic acid and incubated at 37° C. for one hour, then neutralized with Tris buffer to a neutral pH before being tested in the CLB assay, using non-acid treated NAb PCs as a control. As shown in FIG. 2, only PCs 8 and 9 still had more than half of their NAb activity while all the rest had less than 40% NAb activity. Consistent with the acid stability result, when tested in BEAD, acid-resistant clones still had very good inhibition while acid-sensitive clones had either reduced or had completely lost inhibition (FIGS. 3A-3B).

### Example 4

### Optimization of Acid Dissociation of NAb-Drug Complexes

Once the CLB NAb assay format has been determined and the acid stability of the NAb PC is known, the next step is to screen the best acid condition which can maximally dissociate the NAb/drug complex while still preserving NAb activity. A 2-fold dilution of NAb and drug prepared in assay buffer were mixed at 1:1 ratio (10 μL of PC+10 μL of drug) ranging from 156 ng/mL to 2.5 μg/mL for 1 h at 37° C. with shaking at 600 rpm. The samples were then treated with a panel of 12 different acids at varying concentrations and pHs for 15 or 30 mins at RT, followed by addition of biotin-drug to the neutralizing buffer. The final Biotin-drug concentration in the well after mixing the neutralizing buffer with samples was kept at 20 ng/ml. Samples were then added to a pre-blocked SA-MSD plate, and finally washed and detected with sulfo-tagged target protein (0.25 μg/mL). Two acid-stable and 2 acid-sensitive NAb PCs were included in the assessment to assure that the conditions selected were optimal for both acid-sensitive and acid-stable NAb PCs. For this experiment we used 30% inhibition as an arbitrary assay sensitivity/cut point. Table 2 shows the concentration of NAb PC required to achieve a ≥30% inhibition following acid treatment for 15 mins at RT. Based on these data, 50 or 100 mM lactic acid is ideal to be able to detect at least 156 ng/ml of all four NAb PCs and to achieve ≥30% inhibition. Of the two incubation times tested, 15 minutes of acid treatment had higher assay sensitivity and was thus chosen (Table 2 and data not shown).

TABLE 2
Screening acids for PABAD.

| | Acid-sensitive PC | | Acid-stable PC | |
|---|---|---|---|---|
| | PC1 | PC2 | PC3 | PC4 |
| | (ng/mL) | (ng/mL) | (ng/mL) | (ng/mL) |
| 200 mM Glycine pH 2.0 | 2500 | 2500 | 625 | 156 |
| 200 mM Glycine pH 2.3 | 625 | 156 | 313 | 156 |
| 200 mM Glycine pH 2.5 | 313 | 156 | 313 | 156 |
| 150 mM Glycine pH 2.3 | 2500 | 156 | 313 | 156 |
| 150 mM Glycine pH 2.5 | 313 | 156 | 625 | 156 |
| 100 mM Glycine pH 2.5 | 156 | 156 | 313 | 156 |
| 300 mM Acetic acid | 313 | 156 | 313 | 156 |
| 50 mM Lactic acid | 156 | 156 | 156 | 156 |
| 100 mM Lactic acid | 156 | 156 | 156 | 156 |
| 150 mM Lactic acid | 313 | 156 | 625 | 156 |
| 200 mM Lactic acid | 1250 | 156 | 313 | 156 |

### Example 5

### Ratio of Biotin-Drug to the Drug in the Sample for the Best NAb Recovery and Assay Sensitivity

The main advantage of using PandA as first step in the current procedure is that all free mAb drug will remain in the supernatant and eventually be washed away, leaving behind a roughly 1:1 molar

ratio of NAb and drug in the PEG precipitated pellet [12]. This means that the biotin-drug added to the acid-dissociated immune complex to compete for NAb binding can be drastically reduced. To test this hypothesis, and to further optimize the assay readout for improved sensitivity, a range of different concentrations of Biotin-drug and sulfo-tagged target proteins, together with 2 biotin-drug incubation times (90 mins and overnight) were tested. Briefly, a 1:1 ratio of NAb PC and drug, ranging from 41 to 10,000 ng/mL, were mixed and incubated at 37° C. for one hour to form immune complexes. Samples were then dissociated with 50 mM lactic acid and different amounts of biotin-drugs were added to the neutralizing buffer. As shown in Table 3, biotin-drug at 20 or 25 ng/ml with either 0.15 or 0.25 μg/mL sulfo-tag-targets showed very good NAb recovery. Furthermore, increasing the biotin-drug concentration to 38 ng/mL resulted in lower NAb recovery. Hence, 20 ng/ml of biotin-drug and 0.25 μg/mL sulfo-tag-target were chosen as final conditions. Overnight or 90-minute incubations with biotin-drug gave very similar results, thus the 90-minute incubation time was selected (Data not shown).

TABLE 3
Percent of inhibition optimization of biotin-drug
and sulfo-tagged target protein concentration

| | Sulfo-tagged target protein | | | Sulfo-tagged target protein | | |
|---|---|---|---|---|---|---|
| PC + | (0.25 μg/mL) | | | (0.15 μg/mL) | | |
| Drug (1:1) | Biotin-drug, ng/mL | | | Biotin-drug, ng/mL | | |
| (ng/mL) | 38 | 25 | 20 | 38 | 25 | 20 |
| 10,000 | 76 | 81 | 83 | 76 | 80 | 82 |
| 4,000 | 74 | 78 | 82 | 72 | 79 | 81 |
| 1,600 | 68 | 72 | 74 | 66 | 74 | 72 |
| 640 | 61 | 64 | 71 | 59 | 68 | 67 |
| 256 | 42 | 55 | 60 | 41 | 59 | 56 |
| 102 | 30 | 38 | 47 | 24 | 41 | 38 |
| 41 | 29 | 23 | 28 | 17 | 27 | 20 |
| 0 | 0 | 0 | 0 | 0 | 0 | 0 |

Percent inhibition by NAb PC was assessed for each combination of biotinylated drug and sulfo-tagged target protein concentration. Data for one of the 4 PC clones tested is shown and other three followed a similar pattern.

### Example 6

### Optimization of Minimal Required Dilution (MRD) and PEG Precipitation

We first optimized different PEG concentrations as well as PEG formats (e.g., either PEG or PEG-NaCl). PEG or PEG-NaCl solutions were mixed well with serum samples at 1:1 ratio to give rise to the final PEG concentrations of 3%-6% and incubated at 4° C. overnight without agitation. The next day, samples were centrifuged at 3700 g and washed with the same concentration of PEG solution as used for the previous PEG precipitation step. Data in Table 4 shows that PEG-NaCl not only yielded better NAb recovery but also over a boarder range from 3.5-4.5%, compared to that of PEG. A 4% PEG-NaCl concentration was selected for all future experiments.

TABLE 4
Optimization of NAb-drug complex precipitation

| PC | | | | | | | | | | | | | | |
|---|---|---|---|---|---|---|---|---|---|---|---|---|---|---|
| Conc. | PEG | | | | | | | PEG-NaCl | | | | | | |
| (ng/mL) | 3% | 3.5% | 4% | 4.5% | 5% | 5.5% | 6% | 3% | 3.5% | 4% | 4.5% | 5% | 5.5% | 6% |
| 1,000 | 43 | 34 | 30 | 12 | −4 | −5 | −1 | 41 | 54 | 47 | 40 | 20 | 4 | 4 |
| 600 | 47 | 30 | 23 | 4 | −14 | −10 | −3 | 41 | 57 | 51 | 46 | 22 | −1 | 0 |
| 500 | 44 | 33 | 25 | 3 | −14 | −10 | 2 | 46 | 57 | 52 | 49 | 22 | 0 | 3 |
| 400 | 44 | 36 | 19 | −1 | −15 | −7 | −4 | 47 | 57 | 48 | 41 | 23 | −3 | −6 |
| 300 | 47 | 27 | 15 | −4 | −13 | 0 | 6 | 49 | 54 | 48 | 38 | 11 | −1 | 1 |
| 0 | 0 | 0 | 0 | 0 | 0 | 0 | 0 | 0 | 0 | 0 | 0 | 0 | 0 | 0 |

Percent inhibition of binding of sulfo-tagged CD27 with biotinylated drug by NAb was assessed for each precipitation condition. % PEG 8000 (column 2-7), % PEG-NaCl (column 8-14). Data for one of the 2 PC clones tested is shown.

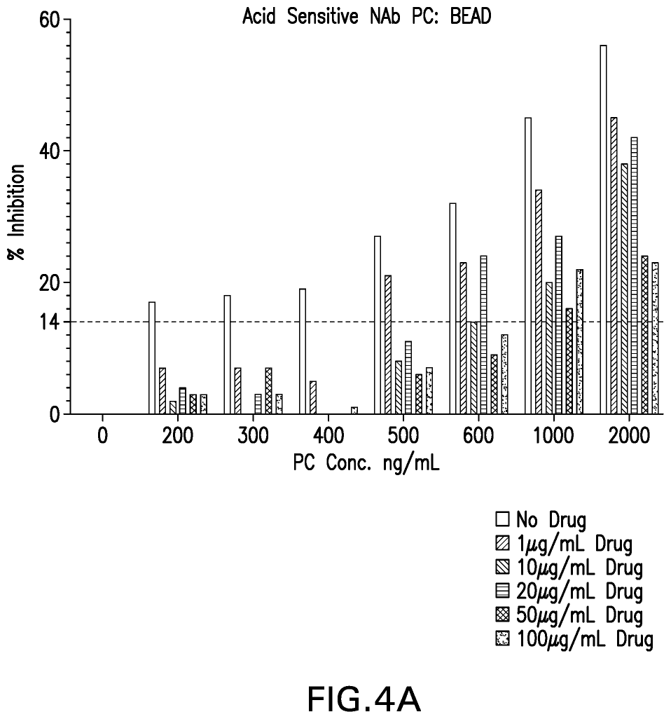

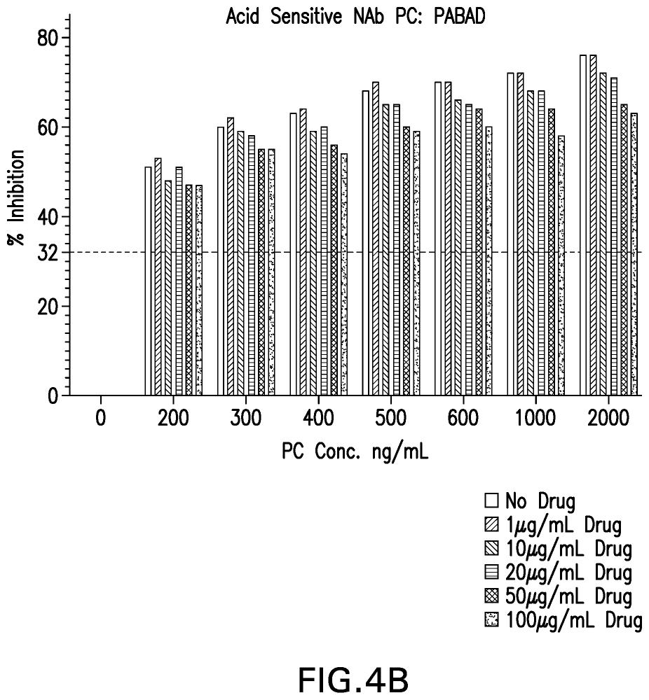

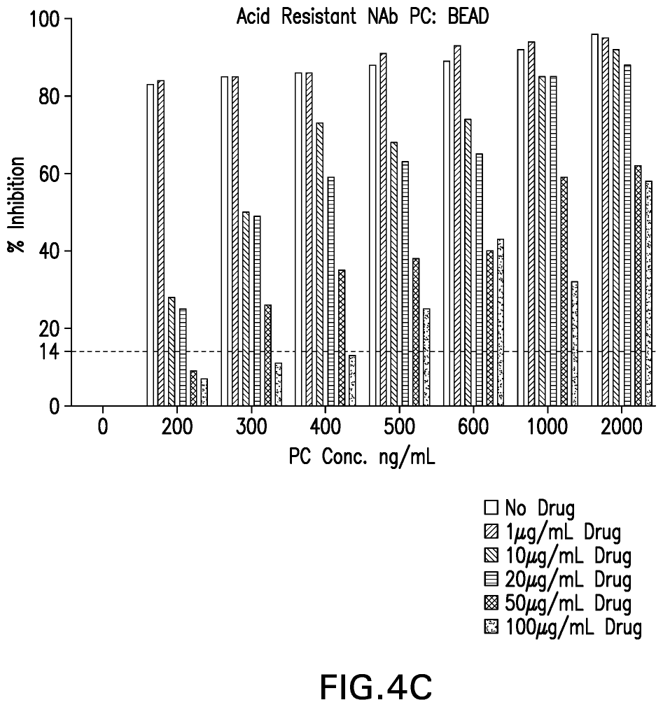

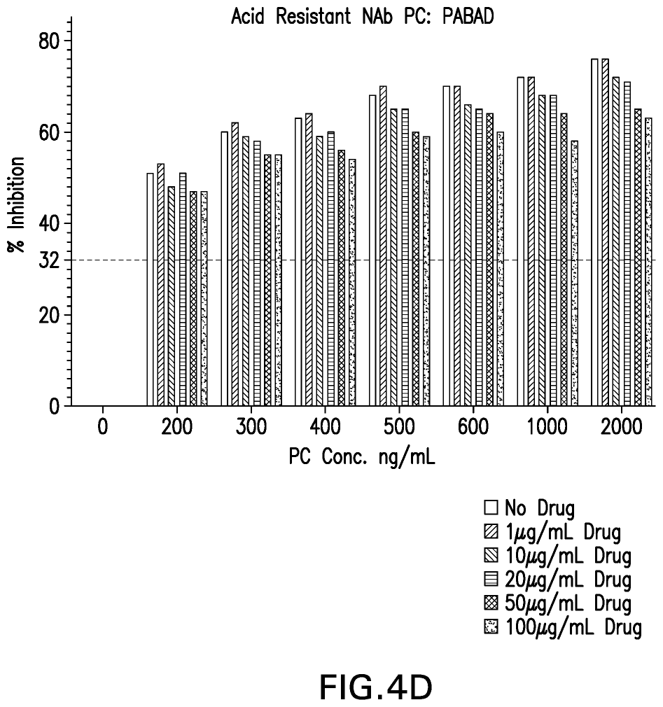

We also tested different MRDs with a panel of 10 individual normal human serum samples, keeping the same concentration of NAb PC. As in previous experiment, 30% inhibition was used as an arbitrary assay sensitivity/cut point. As shown in Table 5, samples from all individual donors with 50% serum (50 μL serum+50 μL PEG-NaCl) showed ≥30% inhibition with ≤500 ng/ml of PC, with most donors requiring only ≤256 ng/mL. Carrying out a further dilution of the serum to 25% did not improve the assay sensitivity. In addition, when 76 individual serum samples were tested at a MRD of 50% as the assay cut point, the standard deviation was relatively small and gave a final cut point of 0.68 (signal/noise ratio) and 32% inhibition (1-signal/noise ratio) (FIGS. 4B and 4D). Hence, 50% serum was selected for future experiments.

TABLE 5
PABAD Assay Selectivity (NAb PC
conc to achieve 30% inhibition)

| | PC Conc in | PC Conc in |
|---|---|---|
| | 50% Serum | 25% Serum |
| Subject | (ng/mL) | (ng/mL) |
| 1 | 500 | 1000 |
| 2 | 205 | 262 |
| 3 | 500 | 512 |
| 4 | 131 | 410 |
| 5 | 256 | 262 |
| 6 | 256 | 262 |
| 7 | 131 | 262 |
| 8 | 131 | 262 |
| 9 | 131 | 328 |
| 10 | 131 | 328 |

Serum from 10 individual healthy donors were tested with determine the matrix interference on assay response. NAb PC concentration to achieve 30% inhibition was used as an estimate for effect of the matrix on assay selectivity.

### Example 7

### Compatibility of Acid Sensitive NAb PCs Using Either BEAD or PABAD

With all the critical steps established including MRD, PEG-NaCl concentration, acid and the amount of biotin-drug and sulfo-tagged drug targets of PABAD optimization, we next compared BEAD and PABAD assays directly with all 10 NAb PCs. As shown in FIGS. 3A-3B, 4 out of 10 NAb PCs completely lost NAb activity with BEAD extraction when NAb PCs were tested at 313 ng/ml. When tested with the PABAD method, however, 9 out of the same 10 NAb PCs had >50% recovery with only one PC demonstrating lower than 40% recovery. These results demonstrate that the PABAD assay is more compatible with acid sensitive NAb PCs, while also maintaining good compatibility with acid-resistant NAb PCs.

### Example 8

a. Assay Sensitivity and Drug Tolerance of PABAD and BEAD in a Therapeutic Drug (First Therapeutic Drug/Drug 1)

Having demonstrated that the PABAD method has broader compatibility with both acid-resistant and acid-sensitive NAb PCs, we then picked two NAb PCs, one acid-sensitive and one acid-resistant, to further compare sensitivity and drug tolerance using both the PABAD and BEAD methods. The NAb PC concentration ranging from 0.2 to 2 μg/mL was spiked into pooled normal human serum together with 0, 1, 10, 20, 50 and 100 μg/mL of drug. Samples were incubated at 37° C. for one hour to form immune complexes.

The cut point was determined by testing 76 normal human serum samples multiple times either with the BEAD or PABAD assays and using the formula of mean minus 1.645-times the standard deviation to obtain the 95th percentile, as recommended. The cut point for BEAD and PABAD assessed this way was 14% and 32% inhibition, respectively, as indicated by the horizontal dashed line in FIGS. 4A-4B.

As shown in FIG. 4A, when this acid sensitive NAb PC was tested with the BEAD method, in the absence of any drug, the sensitivity was good, at around 200 ng/ml. When the drug is present however, even at 1 μg/mL, the sensitivity drops to 500 ng/mL. When the drug is above 50 μg/mL, only 1000 ng/mL NAb can be detected. This is in sharp contrast to the PABAD method as shown in FIG. 4B, where sensitivity is still very good at 200 ng/ml even in the presence of the highest drug concentration tested at 100 μg/mL.

As shown in FIG. 4C, acid resistant NAb PCs had much better compatibility with the BEAD method, so that 200 ng/mL NAb can still be detected in the presence of 20 μg/mL drug. Only at higher drug concentrations of 50 and 100 μg/mL did the sensitivity dropped to 300 and 500 ng/ml, respectively. Interestingly, for the acid-resistant NAb PC tested here, the PABAD method results in maximal assay sensitivity of 200 ng/mL even at the highest drug level at 100 μg/mL, very similar to that of the acid-sensitive NAb PC (FIG. 4D). Thus, the PABAD method demonstrated superior sensitivity and drug tolerance for both acid-sensitive and acid-resistant NAb PCs.

b. Assay Sensitivity and Drug Tolerance of PABAD in Different mAb Therapeutic Drug (Drug 3)

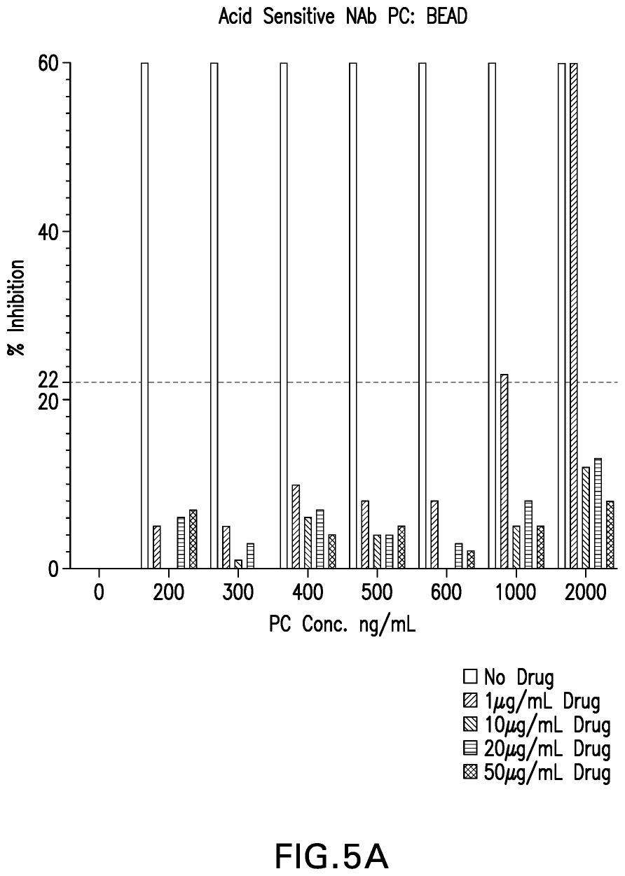

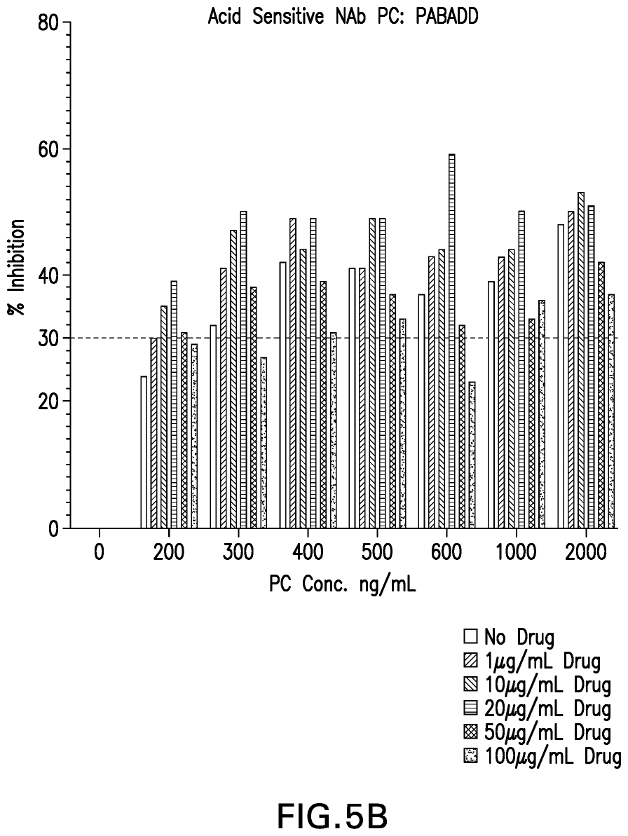

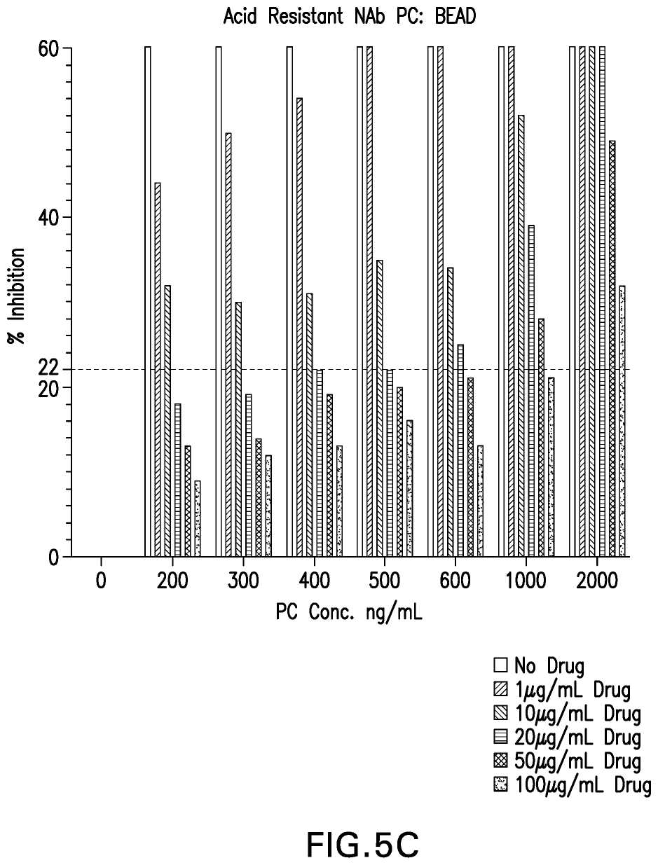

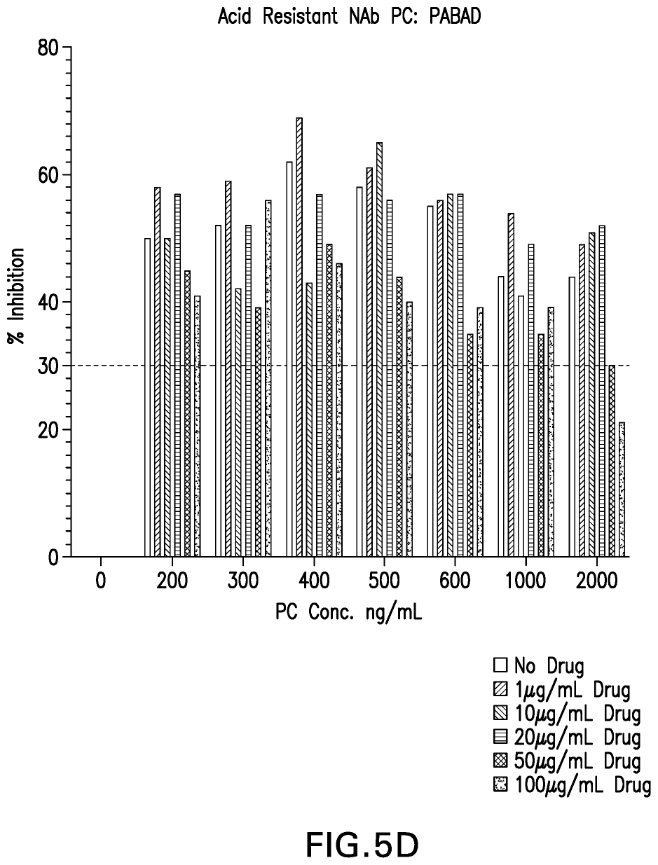

We determined the ease of application of PABAD for the assessment of neutralizing antibodies in other biotherapeutics. We compared sensitivity and drug tolerance of PABAD and BEAD for a second monoclonal antibody therapeutic drug (drug 2) using two neutralizing antibodies, one acid stable and one acid sensitive respectively. Similar to the previous assessment, serially diluted NAb PCs ranging from 0.05 to 2 μg/mL were spiked into pooled normal human serum in the presence of 0, 1, 10, 20, 50 and 100 μg/mL of drug followed by immune complex formation at 37° C. for 1 h. As previously, the CP assessment was carried out using individual serum from 76 naïve subjects on multiples days with different plate layouts. FIGS. 5A-5D showcase the sensitivity and drug tolerance of neutralizing antibody assessment using PABAD or BEAD method for acid sensitive and resistant PC for drug 2.

In the absence of any free drug in the samples, BEAD method showed very high sensitivity for both assay sensitive and resistance NAb PCs (FIGS. 5A and 5C). However, in the presence of the drug the assay sensitivity of acid sensitive PC dropped drastically (FIG. 5A). As expected, acid resistant NAb PC indicated better responses in BEAD assay although the drug tolerance was still in the range of 20 μg/mL. PABAD method on the other hand showed better compatibility with both acid sensitive and acid resistant NAb PC (FIGS. 5B and 5D). In addition to better compatibility with both PCs, PABAD method indicated higher drug tolerance up to 50 μg/mL and 100 μg/mL of free drug for sensitive and resistant NAb PC respectively. Unexpectedly, in the absence of free drug in the samples, PABAD assay sensitivity was lower than BEAD method for both PCs. Anyhow, the PABAD method worked well for the assessment of NAb in drug 2 and the data supports the justification that the method is applicable to NAb assessment in other monoclonal antibody therapeutics.

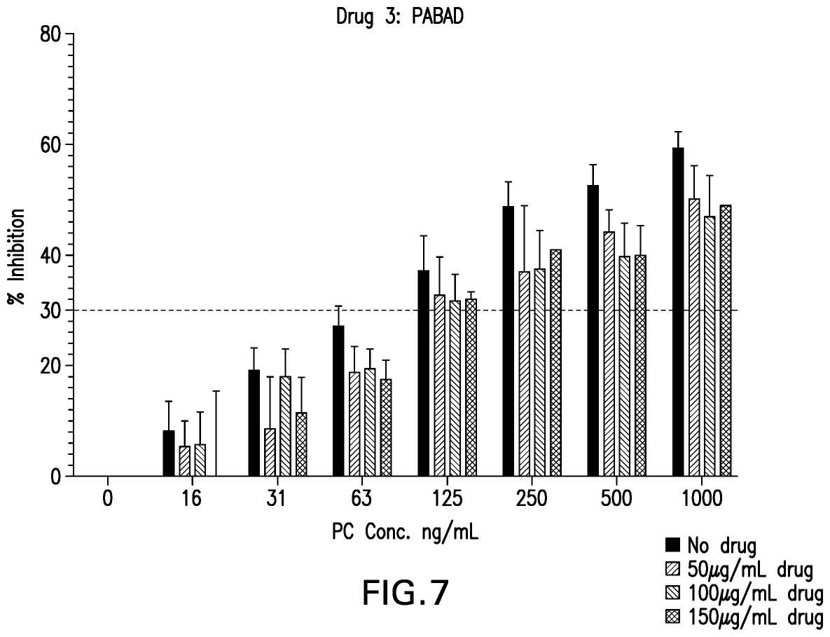

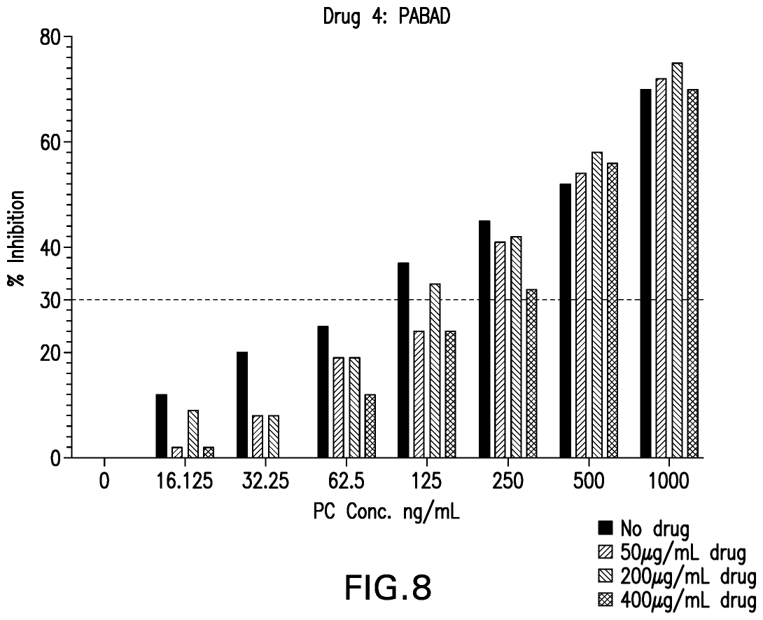

We also measured sensitivity and drug tolerance of PABAD for a third monoclonal antibody therapeutic drug (drug 3), and a fourth monoclonal antibody therapeutic drug (drug 4), similarly to drug 2. PABAD method showed great compatibility for both drug 3 and drug 4, as shown in FIGS. 7 and 8. Maximal assay sensitivity was between 125-250 ng/ml for both drug 3 and drug 4. Drug tolerance was up to 150 μg/mL and 200 μg/mL of free drug for drug 3 and drug 4, respectively.

### Discussion (Examples 1-8)

Drug interference is one of the biggest challenges for NAb assays, especially for mAb pipelines in oncological indications. Acid dissociation and biotinylated-drug extraction has been widely used for both plate- and bead-based pretreatment procedures. Despite mostly successful applications, acid dissociation can fail NAb assays by causing NAb PC denaturation, drug target

releasing hence worsening target interference and incompatibility with PEGylated biologics [10]. In the case of NAb PC denaturation due to acid sensitivity, either milder acid or shorter acid treatment conditions can be screened, or worst case, a new acid resistant NAb PC has to be regenerated and screened. In ADA assays, drug interference is usually easier to overcome, compared to NAb assays, due to the bridging format for most ADA assays. However, when drug levels are very high, a simple acid dissociation may not be enough to overcome drug interference even for an ADA assay [12]. More recently, a new method to overcome drug interference in ADA assays called PEG precipitation and acid dissociation (PandA) has been used [12]. While using PEG to selectively precipitate immune complexes has been known for a long time [13, 14], Zoghbi et al., the first publication describing PandA applies PEG to selectively precipitate immune complex, so that all free drugs in the supernatant can be washed away, leaving behind a 1:1 ratio of ADA:drug immune complexes. This immune complex is then briefly dissociated and coated to an MSD plate under acidified conditions. Ruthenium-labeled drugs are then added and bound on plate-absorbed ADAs to give signal readouts.

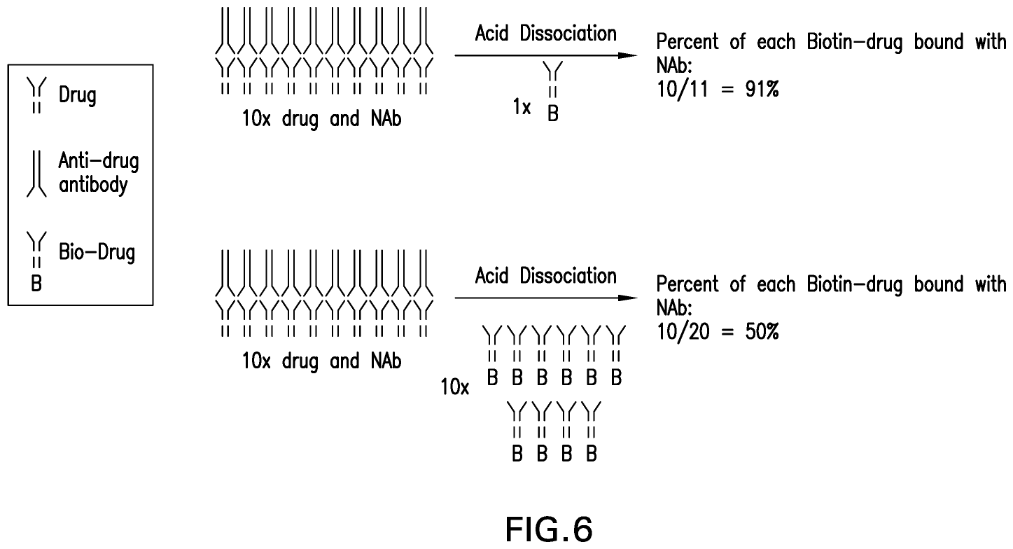

In the PABAD method, one of the advantages is that labeled-drug is used not only to compete for NAb binding but also in the final competitive ligand binding assay. In the BEAD assay, theoretically, to have better competition and better recovery of NAb, biotin-drug needs to be added to a level greater than that of the drug level in the sample. In our experience, we usually start at a 1:1 ratio of biotin-drug to sample drug and sometime need to go as high as 3:1 to achieve good drug tolerance. It is not unusual for mAb biotherapeutics in oncological indications to have sample drug levels in hundreds of micrograms/mL. If the sample contains 100 μg/mL of mAb drug and a starting sample volume of 100 μl is used for BEAD treatment, then 10-30 μg of biotin-drug is needed for each well, which is 1-3 mg for a full 96-well plate. Because of the high amount of biotin-drug used, the amount of magnetic beads needed to pull down these biotin-drug needs to be increased accordingly to make sure all biotin-drug will be pulled down. This is not required for the PABAD method disclosed or claimed herein. And counterintuitively, we found that adding more biotin-drug actually decreased the sensitivity. This is because the percentage of one drug molecule to be occupied by NAb equals to (total NAb)/(sample drug precipitated+biotin-drug added)*100%, regardless of whether it is free drug or biotin-drug. This is illustrated in FIG. 6. Suppose there are 10 NAbs and 10 sample drugs in the precipitated pellet, when I biotin-drug is added, then the chance that each drug, sample drug or biotin-drug, will be occupied by NAb is (10 NAb)/(11 total drug)*100%=90.9%. If 10 biotin-drugs are added, however, now the chance for each drug to be bound by NAb is reduced to (10 NAb)/(20 total drug)*100%=50%. Because of this we can use a concentration as low as 10-20 ng/mL of biotin-drug, which is 0.5-1.0 μg for a full 96-well plate, compared to the 1-3 mg of biotin-drug needed in a typical BEAD assay, which is 1000-fold lower. In addition, no magnetic beads and no 2nd acid dissociation step is needed.

In summary, the disclosed or claimed PABAD method eliminates the harsh and long first acid dissociation in BEAD, uses 1/1000 of the biotin-drug concentration and eliminates magnetic beads, so that acid sensitive NAb species are preserved in a much-simplified procedure. In addition, this pretreatment makes it possible that the same sample goes through PEG precipitation and acid dissociation, then part of the sample can be tested in an ADA assay while the rest of the sample can be neutralized and incubated with biotin-drug at 4° C. Since only a small volume is needed for an ADA assay, screening and confirmatory assays can be done side-by-side. This may seem to increase the confirmatory ADA testing sample numbers, however, the time saved on pulling out and preparing the samples, PEG precipitation overnight etc. could be well worth it. Once ADA results are known, positive samples are then loaded onto SA-MSD to complete the NAb testing. This is a truly an all-in-one approach for immunogenicity testing for mAb biotherapeutics.

In cases where cell-based functional or cell-based binding assays are needed, more biotin-drug in combination with magnetic beads will be needed after PandA precipitation to better compete with the drug in the pellet and recover as much NAb as possible. This will still be better than BEAD treatment because it will only have two shorter and milder acid treatments and less biotin-drug will be needed to just compete with precipitated drug.

### REFERENCES

1. Truong T G, Kennedy L B, Patel S P. 25 Years of Adjuvant Therapy in Melanoma: A Perspective on Current Approvals and Insights into Future Directions. Curr Oncol Rep. 2022; 24 (4): 533-42. doi: 10.1007/s11912-022-01232-0.
2. Cetin B, Wabl C A, Gumusay O. Reshaping Treatment Paradigms for Advanced Renal Cell Cancer Patients and Improving Patient Management: Optimal Management for Renal Cell Cancer Patients. Curr Treat Options Oncol. 2022. doi: 10.1007/s11864-022-00966-0.
3. Hogner A, Moehler M. Immunotherapy in Gastric Cancer. Curr Oncol. 2022; 29 (3): 1559-74. doi: 10.3390/curroncol29030131.
4. Dodge R, Daus C, Yaskanin D. Challenges in developing antidrug antibody screening assays. Bioanalysis. 2009; 1 (4): 699-704. doi: 10.4155/bio.09.55.
5. FDA. Immunogenicity Testing of Therapeutic Protein Products—Developing and Validating Assays for Anti-Drug Antibody Detection. 2019.
6. Casadevall N. Antibodies against rHuEPO: native and recombinant. Nephrol Dial Transplant. 2002; 17 Suppl 5:42-7. doi: 10.1093/ndt/17.suppl_5.42.
7. Xu W, Cummings J, Sank M, Juhel M, Li X, Gleason C, et al. Development and validation of a functional cell-based neutralizing antibody assay for ipilimumab. Bioanalysis. 2018; 10 (16): 1273-87. doi: 10.4155/bio-2018-0109.
8. Xu W, Sank M, Cummings J, Carl S, Juhel M, Gleason C, et al. Bead-extraction and heat-dissociation (BEHD): A novel way to overcome drug and matrix interference in immunogenicity testing. J Immunol Methods. 2018; 462:34-41. doi: 10.1016/j.jim.2018.08.003.
9. Lofgren J A, Wala I, Koren E, Swanson S J, Jing S. Detection of neutralizing anti-therapeutic protein antibodies in serum or plasma samples containing high levels of the therapeutic protein. J Immunol Methods. 2006; 308 (1-2): 101-8. doi: 10.1016/j.jim.2005.10.007.
10. Wu B, Schnarr M, Devlin J L, Brown S, Yang T Y. Approaches to improve drug tolerance and target tolerance in the assessment of neutralizing anti-drug antibodies. Bioanalysis. 2019; 11 (22): 2061-74. doi: 10.4155/bio-2019-0184.
11. Xiang Y, Parng C, Olson K, Seletskaia E, Gorovits B, Jani D, et al. Neutralizing Antibody Assay Development with High Drug and Target Tolerance to Support Clinical Development of an Anti-TFPI Therapeutic Monoclonal Antibody. AAPS J. 2019; 21 (3): 46. doi: 10.1208/s12248-019-0320-3.
12. Zoghbi J, Xu Y, Grabert R, Theobald V, Richards S. A breakthrough novel method to resolve the drug and target interference problem in immunogenicity assays. J Immunol Methods. 2015; 426:62-9. doi: 10.1016/j.jim.2015.08.002.
13. Harkiss G D, Brown D L. Detection of immune complexes by a new assay, the polyethylene glycol precipitation-complement consumption test (PEG-CC). Clin Exp Immunol. 1979; 36 (1): 117-29.
14. Ohlson S, Zetterstrand K. Detection of circulating immune complexes by PEG precipitation combined with ELISA. J Immunol Methods. 1985; 77 (1): 87-93. doi: 10.1016/0022-1759 (85) 90186-3.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method for determining the presence of a neutralizing-antibody (NAb) of a drug in a sample from a subject having received the drug, the method comprising the following steps:
a) contacting the sample with the drug in an amount sufficient to bind any anti-drug antibody (ADA) not already bound to the drug in the sample;
b) precipitating any bound ADA/drug complexes by contacting the sample in step a) with polyethylene glycol (PEG) to yield a ADA/drug complex precipitate;
c) dissociating the ADA/drug complex precipitate with a mild acid solution to yield a mixture comprising free drug and free ADA;
d) contacting the mixture in step c) with the drug having a label to allow the labeled drug to bind to the free ADA to yield labeled ADA/drug complexes;
e) immobilizing the labeled ADA/drug complexes from step d) on an affinity surface, wherein the affinity surface is coated with an affinity molecule for the labeled drug;
f) isolating any NAb/drug complexes from the ADA drug complexes by contacting the immobilized labeled ADA/drug complexes with the drug target having a label to allow the labeled drug target to bind to the drug in the immobilized labeled ADA/drug complexes that are not the NAb/drug complexes, and excess labeled drug target to be washed away;
g) determining the level of the labeled drug target bound to the immobilized labeled ADA/drug complexes;
h) performing steps a) to step g) of the method on a negative control (NC) that does not contain ADA and/or NAb; and
i) comparing the level of the labeled drug target bound to the immobilized labeled ADA/drug complexes in step g) performed with the sample to the level of the labeled drug target bound to the immobilized ADA/drug complexes measured in step g) performed with the NC;
wherein a lower level of the labeled drug target bound to the immobilized labeled ADA/drug complexes in the sample as compared to the level of the labeled drug target bound to the immobilized labeled ADA/drug complexes in the NC indicates the presence of NAb in the sample.

2. The method of claim 1, wherein the drug label and its affinity molecule used in step d) and step e), respectively, are selected from the group consisting of: (i) biotin and streptavidin, (ii) biotin and avidin, (iii) biotin and captavidin, (iv) protein A and immunoglobulin, (v) protein G and immunoglobulin, and (vi) glutathione-S-transferase (GST) and gluthione.

3. The method of claim 1, wherein the drug target label is selected from the group consisting of: (i) a sulfo-tag label, (ii) a chemiluminescent label, (iii) an electrochemiluminescent label, (iv) a radioactive isotope, (v) a fluorescent label, and (vi) an enzyme label.

4. The method of claim 1, wherein the drug label and its affinity molecule used in step d) and step e), respectively, are biotin and streptavidin.

5. The method of claim 1, wherein the labeled drug target is an electrochemiluminescent sulfo-tag label.

6. The method of claim 1, further comprising the step of comparing the level of the labeled drug target bound to the immobilized labeled ADA/drug complexes in step g) performed with the sample to a cut point generated by determining the level of the labeled drug target bound to the immobilized ADA/drug complexes measured in step g) performed with a positive control having Nabs (PC).

7. The method of claim 1, wherein the affinity surface of step e) is an affinity plate.

8. The method of claim 1 wherein the PEG of step b) is provided in a low molecular weight PEG-NaCl solution having a concentration of PEG from about 1% to 8%.

9. The method of claim 1, wherein the PEG of step b) is provided in a PEG-NaCl solution having a concentration of PEG from about 4-5%.

10. The method of claim 1, wherein the PEG is PEG8000.

11. The method of claim 1, further comprising dissociating the NAb/drug complexes from step f) with a mild acid solution to yield a mixture comprising free drug and free NAb, and evaluating the activity of said NAb in a cell-based binding or functional assay.

12. The method of claim 1, wherein the dissociation with the mild acid solution is carried out for a period of less than 60 minutes.

13. The method of claim 6, wherein the dissociation with the mild acid solution is carried out for a period of about 15 minutes.

14. The method of claim 11, wherein the method is carried out in a 96-well plate.

15. The method of claim 14, wherein the amount of labeled drug used per well of the 96-well plate in step d) is less than 10 ng.

16. The method of claim 1, wherein the amount of the drug having a label used per well of the 96-well plate in step d) is less than 1 ng.

17. The method of claim 1, wherein the drug is a therapeutic antibody drug selected from the group consisting of:
a. an anti-CD27 antibody or an antigen binding fragment thereof;
b. an anti-LAG3 antibody or an antigen binding fragment thereof;
c. an anti-TIGIT antibody or an antigen binding fragment thereof;
d. an anti-VISTA antibody or an antigen binding fragment thereof;
e. an anti-BTLA antibody or an antigen binding fragment thereof;
f. an anti-TIM3 antibody or an antigen binding fragment thereof;
g. an anti-CTLA4 antibody or an antigen binding fragment thereof;
h. an anti-HVEM antibody or an antigen binding fragment thereof;
i. an anti-CD70 antibody or an antigen binding fragment thereof;
j. an anti-OX40 antibody or an antigen binding fragment thereof;
k. an anti-CD28 antibody or an antigen binding fragment thereof;
l. an anti-PD1 antibody or an antigen binding fragment thereof;
m. an anti-PDL1 antibody or an antigen binding fragment thereof;
n. an anti-PDL2 antibody or an antigen binding fragment thereof;
o. an anti-GITR antibody or an antigen binding fragment thereof;
p. an anti-ICOS antibody or an antigen binding fragment thereof;
q. an anti-SIRPα antibody or an antigen binding fragment thereof;
r. an anti-ILT2 antibody or an antigen binding fragment thereof,
s. an anti-ILT3 antibody or an antigen binding fragment thereof;
t. an anti-ILT4 antibody or an antigen binding fragment thereof;
u. an anti-ILT5 antibody or an antigen binding fragment thereof;
v. an anti-4-1BB antibody or an antigen binding fragment thereof;
w. an anti-NK2GA antibody or an antigen binding fragment thereof;
x. an anti-NK2GC antibody or an antigen binding fragment thereof;
y. an anti-NK2GE antibody or an antigen binding fragment thereof;
z. an anti-TSLP antibody or an antigen binding fragment thereof; and
aa. an anti-IL 10 antibody or an antigen binding fragment thereof.

18. The method of claim 1, wherein the drug is a human or humanized monoclonal antibody drug.

19. The method of claim 1, wherein the drug target is a protein antigen selected from group consisting of TNFα, CD27, PD1, PDL1, PDL2, TIGIT, LAG-3, VISTA, BTLA, TIM3, CTLA4, HVEM, CD70, OX40, CD28, GITR, ICOS, SIRPα, IL10, IL-4R, IL-6R, IL-33, CD20, CD3, IL-33, IL-2, IL-15, IL-18, Feld 1, C5, ANGPTL-3, ACTIVIN A, GDF8, PCSK9, VEGF, Tie-2, and NGFN.

20. The method of claim 1, wherein the drug target is a viral antigen is selected from a group consisting of SARS-COV, RSV, ebola and MERS-COV.

21-22. (canceled)
